(19) United States
(12) Patent Application Publication
(10) Pub. No.: US 2026/0290586 A1
(43) Pub. Date: Sep. 24, 2026

(54) AN INTERACTIVE METHOD FOR SUPPORTING PATIENTS IN THE END OF THEIR LIFE

(71) Applicant: EPILOG END OF LIFE COMPANION LTD, Gimzo (IL)

(72) Inventors: Tal SHAPSA HEIMAN, Tel Aviv (IL); Ofir SHAUMAN, Gimzo (IL); Or BAR-TAL, Tel Aviv (IL); Dan ARIELY, Durham, NC (US); Ron SABAR, Herev Leet (IL)

(21) Appl. No.: 19/472,420

(22) PCT Filed: Apr. 4, 2024
(86) PCT No.: PCT/IL2024/050349

§ 371 Date: Oct. 3, 2025

(30) Foreign Application Priority Data
Apr. 4, 2023 (IL) 301954

(51) Int. Cl. G16H 50/20; G16H 10/20; G16H 10/60; G16H 50/30

(52) U.S. Cl. CPC G16H 50/20; G16H 10/20; G16H 10/60; G16H 50/30

(57) ABSTRACT

The present disclosure provides a method and a system for interacting with a user having a medical condition, including generating interactive content to support the user's decisions, providing recommendations, and suggest palliative care treatments. The typical user is one with special medical conditions, potentially (but not limited to) in a terminal stage, which mostly means in the last couple of years of life. This stage requires a specific set of actions and decision-making processes. The method may apply a data-driven and possibly AI-based methodology based on medical and behavioral principles, with a major focus on palliative care. The method provides adaptable solutions and tools, which are user-specific, based on the user's profile (including their medical, mental, psychological, and economic state) for managing their end-of-life journey. The method considers the user's special condition, particularly their mental and physical states, and to provide an interaction with the user that is intended to progress the user towards one or more goals that are related to end-of-life issues. For example, the method is intended to provide the user with support and guidance and help the user with decision-making and tasks, which in some cases are necessary to accomplish (e.g., filling out insurance forms and writing a will). Moreover, the method also provides suggestions and helpful information to better the user's current condition. The method makes use of several technological solutions, such as formulated decision trees, chatbots, and machine-learning models, which combine different types of data.

Furthermore, the method comprises of theory-based models, one of those is based on the mental capacity theory, which identifies the user's mental strength and the user's mental capacity for taking actions and making decisions. Another model is based on the total pain theory, which identifies the underlying contributors to the user's self-reported pain, including physiological and mental causes.

The models outputs and predictions, the users' input data, and the users' interaction history data (i.e., all data associated with their interaction with the method, as well as their responses to questions output by the method), are used to generate different types of output to the user, as well as to infer various parameters indicating the current state of the user.

### TECHNOLOGICAL FIELD

The present disclosure is in the field of engagement with subject or patients for providing them support and assisting in making decisions.

### BACKGROUND ART

References considered to be relevant as background to the presently disclosed subject matter are listed below:
WO 2016/073479

Acknowledgement of the above references herein is not to be inferred as meaning that these are in any way relevant to the patentability of the presently disclosed subject matter.

### GENERAL DESCRIPTION

The present disclosure provides a method for interacting with a user, including generating interactive content to support the user's decisions, providing recommendations, and suggest palliative care treatments. The typical user is one with special medical conditions, potentially (but not limited to) in a terminal stage, which mostly means in the last couple of years of life. This stage requires a specific set of actions and decision-making processes. The method may apply a data-driven and possibly AI-based methodology based on medical and behavioral principles, with a major focus on palliative care. The method provides adaptable solutions and tools, which are user-specific, based on the user's profile (including their medical, mental, psychological, and economic state) for managing their end-of-life journey. The method considers the user's special condition, particularly their mental and physical states, and to provide an interaction with the user that is intended to progress the user towards one or more goals that are related to end-of-life issues. For example, the method is intended to provide the user with support and guidance and help the user with decision-making and tasks, which in some cases are necessary to accomplish (e.g., filling out insurance forms and writing a will). Moreover, the method also provides suggestions and helpful information to better the user's current condition. The method makes use of several technological solutions, such as formulated decision trees, chatbots, and machine-learning models, which combine different types of data.

Furthermore, the method comprises of theory-based models, one of those is based on the mental capacity theory, which identifies the user's mental strength and the user's mental capacity for taking actions and making decisions. Another model is based on the total pain theory, which identifies the underlying contributors to the user's self-reported pain, including physiological and mental causes.

The models outputs and predictions, the users' input data, and the users' interaction history data (i.e., all data associated with their interaction with the method, as well as their responses to questions output by the method), are used to generate different types of output to the user, as well as to infer various parameters indicating the current state of the user.

Therefore, an aspect of the present disclosure provides a method for interacting with a user, including identifying gaps in current care the user is receiving, generating interactive content for supporting the user decisions, providing recommendations, and suggesting palliative care treatments. The method comprises receiving user-related data, that includes any combination of medical-related data of the user, financial data, the physical conditions in the surrounding or the living area of the user (such as weather, living conditions), psychological data indicative of the psychological condition of the user, social data indicative of the social environment of the user, user interaction/engagement history, interaction/engagement profile history of the user, etc. The method further comprises analyzing said user-related data for generating a user temporal profile. The user temporal profile comprises one or more temporal user scores, affecting said temporal profile, namely a change in the temporal user score may change the user temporal profile. The temporal user scores are calculated based on input of various parameters or features that affect it. The temporal user scores can be also referred as target functions that the method is intended to improve through interaction of the user with a content being output to him/her. The temporal user scores comprises at least one of (1) end of life score indicative of the life expectancy of the user or a user's perception of his/her life expectancy; (2) at least one of mental capacity score indicative of the capacity of the user for facing with or taking decision on a certain topic, and total pain score indicative of the total physical and mental pain that the user is experiencing; (3) user state model indicative of his/her mental capacity and total pain; (4) readiness score of the user to make transition to a different level or location of care (e.g., readiness to receive palliative care, readiness to move to hospice). This may also include expectancy of the physical and mental condition of the user until the end of his/her life. For every score, there may be a calculated confidence level, indicating the level of familiarity with the user and the verified contributing factors to determining the score. The method further comprises selecting an interactive content option from a plurality of interactive content options based on said user temporal profile to improve at least one of said one or more temporal user scores. The plurality of interactive content options are associated with an effect on at least one of said one or more temporal user scores. Namely, the interactive content option is expected to change and probably increase the temporal user score that it has an effect on. The method further comprises outputting said interactive content option to the user or a secondary user, whereas the secondary user is an individual that is related to the user and can assist in providing details of the user or in improving engagement of the user.

In some embodiments, the method comprises recording a response of the user to said output interactive content option and updating at least one of said one or more temporal user scores, thereby updating the user temporal profile. The user-related data comprises the recorded response, namely it is considered one of the inputs that affects the journey of the interactive communication of the user.

In some embodiments of the method, the user-related data comprises user interaction history data, which is the recorded results of the interaction of the user with the output interactive content options, including their responses as well as interaction metadata such as response time.

In some embodiments of the method, the user interaction history data comprises recorded responses of the user to each output interactive content option.

In some embodiments of the method, the user interaction history data comprises engagement profile of the user with each output interactive content option. This includes the response time of the user to the interactive content option, the time of the day of the engagement with the output interactive content option. The engagement profile comprises temporal engagement behavior of the user that indicates the tendency of the user to engage with interactive contents in a certain time of the day, the month, or a certain time after a previous interactive content. The selection of an interactive content option is affected by the temporal engagement behavior. For example, if a user does not tend to engage with content in certain times of the day, the output of the interactive content that is selected will not be output in these times of the day and be postponed to the time of the day in which the user is more active.

In some embodiments of the method, the engagement profile defines a periodical engagement score. The periodical engagement is a score that indicates the level of engagement of the user in a selected period. In some embodiments, the method outputs the selected interactive option only at times that the periodical engagement score is above a certain threshold. The period may be a day, a week, a month, times between output of a selected interactive content, etc.

In some embodiments of the method, the engagement profile comprises recording of the physical engagement profile of the user with said interactive content option when said interactive content option is being presented on a touch screen. Namely, where the user pressed on the presented interactive content, what is the force applied on the touch screen, speed of response, number of presses on the same screen, number of times reviewed (or listened to, in audio/video features), etc.

In some embodiments of the method, the user interaction history data comprises a plurality of data pieces, each associated with a different time stamp, namely a time that indicates when the data piece was received/recorded or the time that this data piece is related to. Each data piece is assigned with a respective weight factor for affecting at least one temporal user scores.

In some embodiments, the method comprises updating said respective weight factor assigned to each data piece based on the respective time stamp of said data piece.

In some embodiments of the method, said updating said respective weight factor comprises applying a time stamp-depended function to said respective weight factor. In other words, each data piece of the history data has a weight factor that affect its effect on one or more temporal user scores that is dependent on the time stamp that is assigned to it. For example, relatively old data pieces can have an effect on a certain temporal user score that is lower than newer data pieces.

In some embodiments of the method, the user related data comprises responses of said secondary user to the output interactive content option.

In some embodiments of the method, the user related data comprises medical data. The medical data may include any medical information of the user, including diagnosis of diseases, drugs subscribed to the user, history of medical procedure, pain levels that the user is experiencing, etc. In some embodiment of the method, said medical data comprises life expectancy data indicative of the life expectancy of the user or the medical condition that is expected until the end of the user's life, e.g. expected deterioration of the physical or medical condition of the user.

In some embodiments of the method, the user related data comprises psychological data. This can include responses to psychological questionnaires, such as questions regarding the user's distress and anger level as well as indirect questions inferring tendency for control, impact of receiving medical information on resilience, and etc. This may also include a report from a psychologist or other caregiver, etc.

In some embodiments of the method, the user related data comprises social data. This can be obtained by a self-report of the user of his/her social condition or any other social-related input. The social data may include family or marriage status, number of children, number of residents in the house, information about involvement of the user in the community, and data extracted from the user profiles from social media.

In some embodiments of the method, the user related data comprises financial data. That may comprise credit score, financial assets of the user, etc.

In some embodiments of the method, the one or more temporal user scores comprise at least one of the following: (1) mental capacity score, indicative of the mental capacity of the user to perform actions or making decisions; (2) medical burden score, indicative of the burden that is associated with the medical condition of the user; (3) total pain score, indicative of the combined or accumulative pain of the user from several pain sources, including one or more of physical pain, social pain, spiritual pain and psychological pain. In some embodiments, the total paint score may include the medical burden score; (4) friends and family support score, indicative of the level of support that the user receives or feels he/she receives or need to receive from friends and family; (5) tendency to share medical condition, indicative of the probability that the user will share details of his/her medical condition if asked on it; (6) tendency to share content, indicative of the probability that the user will share any type of content, not necessarily related to his/her medical condition; (7) tendency to share financial challenges; (8) tendency to consume services, indicative of the probability of the user to consume a service if suggested to him/her; (9) tendency to create content; (10) level of peace (11) level of control, indicative of the user's general sense of control of their situation, and/or level of control in their symptoms (physical or emotional) or otherwise control; (12) urgency to create legacy (13) level of meaning (14) remaining time perception, namely the perception of the user of the time left for him/her until death, which can provide an indication about the intentions of the user to cooperate and to progress some issues before death; (15) level of fear of death; and (16) home treatment trending score, indicative of the desire of the user for receiving home treatment, e.g., the desire of the user for hospice care or home palliative care; (17) level of readiness to change location of care, indicative of member readiness to move from home to institutional setting, or from hospital to home or to institutional setting; (18) level of readiness to receive benefit to support quality of life, indicative of readiness to receive care that is focused on quality of life, such as palliative care, spiritual care or psychosocial support, whether or not such benefit is financed by an insurance entity or else, or paid for in private; (19) tendency to admit to hospital.

In some embodiments of the method, the one or more temporal user scores comprise at least mental capacity score. The mental capacity score reflects the mental fitness of the user to make decisions on his/her own at the present moment or in the near future. This score indicates the capability of the user to deal with certain issues and complex emotional interactions, and the capability of the user to make decisions to improve the quality of his/her life and to conduct an effective journey of the rest of his/her life. By monitoring this score, the method provides an ability to select the interactive content options that deal with certain issues that the user can handle and manage at any given time of the process. The mental capacity score may further indicate the effect of each interactive content option on the progress of the user in one or more different temporal user scores.

In some embodiments of the method, the one or more temporal user scores comprise at least total pain score. The total pain score is a sum of the physiological sources of pain and the mental sources of pain. The physiological sources of pain results from a medical condition that has physiological aspects. The mental sources of pain occur alongside with the physical pain and augment it, and derive from psychological reasons, social reasons, spiritual reasons, and cultural reasons. The calculation of the total pain score is performed by a time-based model that takes into account the time stamp of any input that feeds it. In some embodiments, the total pain score affects the mental capacity score, namely the mental capacity score is constantly affected by the total pain score. The total pain score may indicate the factors that are causing the pain for the user and assist in selecting an interactive content option that may improve the total pain score. In other words, the total pain score affects the selection of the interactive content option to the user so as to result in a decrease of the total pain of the user.

In some embodiments of the method, said selecting comprises selecting an interactive content option that is associated with an effect on one or more desired temporal user scores. In other words, the selection of the interactive content is made in order to obtain an optimal effect or improvement on one or more specific temporal user scores.

In some embodiments of the method, the plurality of interactive content options comprises range-based interactive content options that are allowed for selection upon being in a defined range of one or more temporal user scores. It is to be noted that some range-based interactive content options are only allowed upon reaching a defined range in two or more temporal user scores, each range may be different from the other.

In some embodiments, the method comprises updating the defined range of each of said range-based interactive content based on said user temporal profile, Namely, the range-based interactive content is personalized and may be based or updated according to the user temporal personal profile.

In some embodiments, the method comprises assigning one or more interactive content option weight factors affecting each temporal user scores to at least one interactive content option.

In some embodiments, the method comprises updating said interactive content option weight factors according to said user temporal profile.

In some embodiments, the method comprises updating said interactive content option weight factors using one or more machine-learning algorithms, such as, but not limited to, decision trees, random forest, SVM and LSTM.

In some embodiments, the method comprises assigning a variance parameter to at least one interactive content option. The variance parameter indicative of the variance of interactions of a population of users with said interactive content option. The assignment or the update of one or more interactive content option weight factors comprises applying a variance function dependent on said variance parameter.

In some embodiments of the method, the variance function is further dependent on said user temporal profile. Namely, the variance of the responses for the respective interactive content affects the extent of the effect of the user temporal profile on updating the interactive content option weight factors. For example, if an interactive content option has a relatively low variance of responses by a population of users, the effect of the user temporal profile is relatively low; if an interactive content option has a relatively high variance of responses by a population of users, the effect of the user temporal profile is relatively high.

In some embodiments of the method, the interactive content options comprise questions or questionnaires, that may be in the form of a chatbot.

In some embodiments of the method, the interactive content options comprise recommendations for actions or for services, such as recommendation to take a financial consultancy, palliative treatment suggestion, medical treatment suggestion, suggestion to accept a medical or non-medical benefit, suggestion to transition to hospice, legal advisory, suggestion to have a memory book, suggestions to donate for charity, suggestions for spending meaningful time with friends and family, manage difficult conversation with friends and family, meditation suggestions, suggestion of how to communicate medical wishes to clinical teams, suggestion to sign forms (e.g., advanced care planning documentations) etc.

In some embodiments of the method, the recommendations comprise palliative treatment suggestions. For example, how to manage pain and other symptoms, including emotional symptoms, how to decide on goals of care and how to communicate them to clinical team and friends and family, how to improve comfort of care, etc.

In some embodiments of the method, the interactive content options comprise tasks for the user. This includes, for example, taking some financial decisions, treatment selection, complete questionnaire, complete legal forms and other types of forms, etc. This may also include tasks for improving wellbeing, such as taking a walk in the park, vacation, expressing gratitude, having meaningful conversations with friends and family, etc.

In some embodiments, the method comprises receiving general medical-related data indicative of medical statistics, such as statistics of diseases, medical conditions, hospitalization periods, treatments, medical insurances, etc. The step of selecting an interactive content option is further performed based on said general medical-related data. In other words, generic medical data in combination with the specific user related data affect the user temporal profile and the interactive content option that is selected in order to improve one or more temporal user scores of the user. Optionally, the general medical-related data can also affect directly on the user temporal profile.

In some embodiments, the method comprises receiving location-related data indicative of data related to specific locations, including whether, medical facilities in a certain location, rules and regulations in a specific territory, etc. The step of selecting an interactive content option is further performed based on said location-related data. For example, the selected interactive content option can be a suggestion to receive a certain treatment and the user is directed to a facility that provides such treatment in the vicinity of his/her home. Another example is a suggestion to receive professional legal assistance from a professional in the vicinity of the home of the user. Furthermore, the method can take into account the weather condition in the area of the user to time an output of a certain interactive content option to when there is a good weather. Optionally, the location-related data can also affect directly on the user temporal profile.

Yet another aspect of the present disclosure provides a system for interacting with a user. The system comprises at least one processing circuitry, i.e. a processor or a processing unit, which can be centralized or distributed in several locations. For example, some of the processing power can be in the user's device and some can be in the cloud. The at least one processing circuitry is configured for: receiving user-related data; analyzing said user-related data for generating a user temporal profile, said user temporal profile comprises one or more temporal user scores, said temporal user scores comprises at least one of mental capacity score indicative of the capacity of the user for facing with or taking decision on a certain topic, and total pain score indicative of the total physical and mental pain that the user is experiencing; selecting an interactive content option from a plurality of interactive content options based on said user temporal profile to improve at least one of said one or more temporal user scores, each of the plurality of interactive content options is associated with an effect on at least one of said one or more temporal user scores; and outputting said interactive content option to the user or a secondary user.

In some embodiments, the system further comprises an output unit for outputting said interactive content option. The output unit may be a mobile device, a tablet, a computer, or any other device that can serve for outputting visual and optionally audible content.

In some embodiments of the system, the at least one processing circuitry is configured to perform any of the above-described embodiments of the method or any combination thereof.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1C are flow diagrams of different non-limiting examples of methods for interacting with a user according to aspects of the present disclosure.

FIG. 2 is an illustration exemplifying the logic of the flow of selecting an interactive content option.

### DETAILED DESCRIPTION

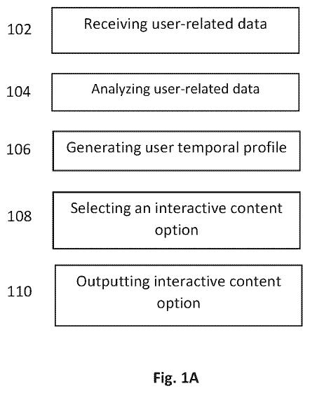

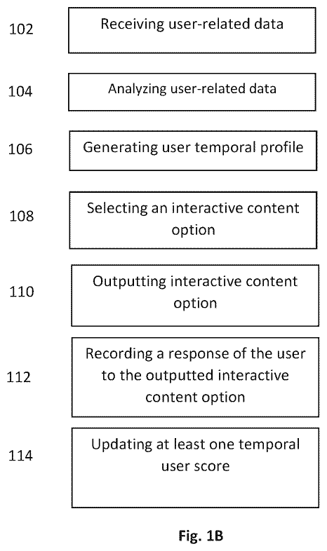

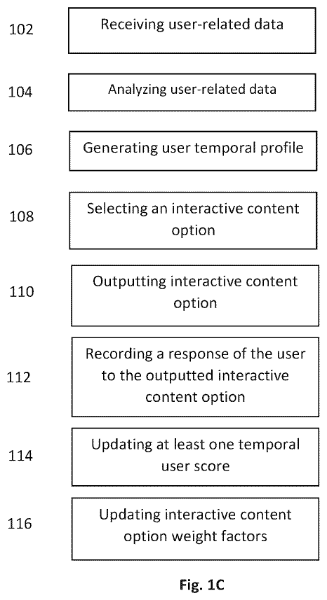

Reference is made to FIG. 1A-1C, which are flow diagrams of non-limiting examples of a method for interacting with a user, providing recommendations and supporting decision-making. FIG. 1A exemplifies a method that comprises, after initiating interaction with the user, receiving user-related data 102, which may include medical data, psychological data, social data, financial data. The user-related data is intended to be used for generating a unique profile of the user that characterize him/her with respect to the mental and physical state he/she is in, and the decisions he/she needs to make in financial aspects, legal aspects, and palliative treatment aspects. The data can be received by an input of the user through pre-prepared forms, response to AI-generated questions, or by an input from other sources, such as medical record received from a medical institution, insurance company, government database, legal document from a legal firm, etc. The method may comprise also receiving general medical-related data indicative of medical statistics, such as statistics about diseases, medical conditions, treatments, etc. Furthermore, the method may comprise receiving location-related data indicative of data related to specific locations, such as weather conditions, medical facilities, relevant laws and regulations, etc. Out of these general medical-related data and location-related data there may be data relevant to the specific user and this data can be treated also as a user-related data. Therefore, the general medical-related data and the location-related data can affect the user temporal profile. The method further comprises analyzing the user-related data 104 for generating a user temporal profile 106. The analysis of the data may include several steps including standardization of the data and processing of the data. These steps may include the utilization of Natural Language Processing, stratification of the data or other known methods for processing input data.

The user temporal profile comprises one or more temporal user scores, which comprises at least one of: (1) end of life score indicative of the life expectancy of the user or a user's perception of his/her life expectancy; (2) at least one of mental capacity score indicative of the capacity of the user for facing with or taking decision on a certain topic, and total pain score indicative of the total physical and mental pain that the user is experiencing; (3) user state model indicative of his/her mental capacity and total pain. These scores are used to characterize the user and to determine the required engagement with the user in order to improve at least one temporal user score so as to bring the user to a condition that he/she can make decisions and take actions on several aspects relating to the end of his/her life including financial and legal aspects (such as will, insurance, etc.) and palliative aspects (such as home treatment).

The temporal user scores may also comprise at least one of the following: mental capacity score, medical burden score, total pain score, friends and family support score, tendency to share medical condition, tendency to share content, tendency to share financial challenges, tendency to consume services, tendency to create content, level of peace, level of control, urgency to create legacy, level of meaning, remaining time perception, level of fear of death, and home treatment trending score.

The method further comprises selecting an interactive content option 108 from a plurality of interactive content options based on said user temporal profile to improve at least one of said one or more temporal user scores. The interactive content selection can be performed based on the general medical-related data and the location-related data. Each of the plurality of interactive content options is associated with an effect on at least one of said one or more temporal user scores. Thus, the selection of an interactive content option 108 comprises selecting an interactive content option that is associated with an effect on one or more selected temporal user scores. The selected temporal scores are temporal scores that at a certain time point in the interaction with the user are selected to be in focus and the interaction at this time point is performed in order to improve these temporal scores. Namely, the selection of the interactive content option is being done by considering the optimal selection that will achieve the optimal effect on the temporal user scores. The selection of the interactive content option or even the selected temporal score to be in focus may be performed also based on maximizing an engagement score that is determined by the engagement profile of the user. Based on the engagement profile that is collected on the user, the selection of the interactive content option may be biased to an option that the user will tend to engage with, rather than other interactive content options that may even improve more the selected temporal score that is in focus. Another implementation of the engagement profile is to time the selected interactive content option to a time that will probably obtain the maximum engagement of the user with the content being output. Therefore, an engagement profile layer of the user may affect the content being selected and the timing of the output according to a time window that predicts maximal engagement by the user.

The interactive content options also comprise range-based interactive content options that are allowed for selection only upon being in a defined range of one or more temporal user scores. Namely, in order to allow the selection of range-based interactive content options, one or more temporal scores are required to be in a certain range. The defined range of each of the range-based interactive content options is updated based on the user temporal profile.

The method further comprises outputting the interactive content option 110 to the user, or to a secondary user, which typically has some form of relation to the user. The interactive content option can be questions or questionnaires, tasks for the user, recommendations for actions or for services, such as palliative treatments.

Reference is now being made to FIG. 1B. The method exemplified in FIG. 1B differs from that of FIG. 1A by further comprising recording a response of the user to the output interactive content option 112 and updating at least one of the temporal user scores 114, which also affects the user temporal profile. The update of the temporal score also includes updating the confidence level that indicates the level of familiarity with the user and the verified contributing factors to determining the score. Namely, after each engagement of the user with the interactive content option, the confidence of the specific temporal score increases and also the confidence on the building blocks factors that are associated with determining the temporal score increases. Thus, the user-related data 102 also comprise user interaction history data, which include the recorded responses of the user to each output interactive content option, as well as an engagement profile of the user with each output interactive content option. The engagement profile comprises for example the user's response time to the output interactive content option with respect to the time the content was output (i.e., the time it the user to response after the interactive content is output), the time of day the interaction takes place, etc. Also, the engagement profile comprises recordings of the physical engagement profile of the user with said interactive content option when the interactive content option is being presented on a touch screen, such as the force applied by the user on the touch screen. It is to be noted that the recorded response may be the responses of the secondary user to the output interactive content option. Thus, the user related data may also comprise responses of said secondary user to the output interactive content option. The user interaction history data also comprises a plurality of data pieces, each associated with a different time stamp wherein each data piece is assigned with a respective weight factor for affecting at least one temporal user score. These weight factors are updated based on the respective time stamp of the respective data piece, by applying a time stamp-depended function to the respective weight factors. This allows to consider the time stamp of the recorded data, when adjusting the effect of each data pieces on one or more temporal user scores, through their respective weight factors. For instance, an old data piece related to the current psychological state of the user, could be less significant and by that with a lower effect on a certain relevant temporal user score which considers the user psychological state.

Reference is now being made to FIG. 1C, which exemplifies a method that differs from that of FIG. 1B by further comprising updating interactive content option weight factors 116. It is to be understood that the interactive content options are assigned with one or more interactive content option weight factors affecting each temporal user scores. The interactive content option weight factors are updated constantly based on the user temporal profile. Typically, the process of updating the interactive content option weight factors is performed by using one or more machine-learning algorithms that receive inputs from parameters of the user temporal profile, such as inputs of the current temporal scores of the user. The effect of the user temporal profile on the update of the interactive content option weight factors is further dependent on a variance parameter, indicative of the variance of interactions of a population of users with said interactive content option, that is assigned to the interactive content options. Therefore, assigning and/or updating the interactive content options weight factors also comprises applying a variance function dependent on said variance parameter. The variance function is further dependent on the user temporal profile, for the purpose of allowing the variance to influence the magnitude of the effect of the user temporal profile on updating the interactive content option weight factor.

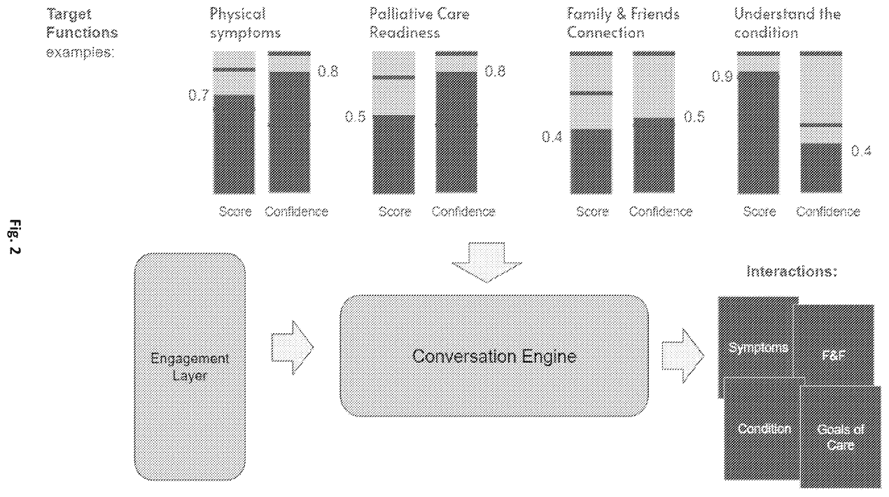

Reference is now made to FIG. 2, which is a schematic illustration of the process of decision making for the next interactive content option to be output. The figure exemplifies it by four target functions, namely four temporal scores of the user. For each target function there is a range of target scores. If the score of the user is not in the target range, the conversation engine determines what is the next interactive content option that should be selected in order to get the score of the user closer or in the target range. The engagement layer comprises information of the engagement profile of the user along a selected period of time, e.g. a day, a week, a month, or a certain period of time following an output of a previous content option. Therefore, according to the engagement layer, in order to maximize the engagement of the user with the selected interactive content option, the output of the selected interactive content option is timed. Therefore, after a selection of an interactive content option to be output to the user, the engagement layer controls the timing of outputting the selected interactive content option.

1-33. (canceled)

34. A method for interacting with a user having a medica condition, comprising:
receiving user-related data;
analyzing said user-related data for generating a user temporal profile, said user temporal profile comprises one or more temporal user scores, said temporal user scores comprises at least one of mental capacity score indicative of the capacity of the user for facing with or taking decision on a certain topic, and total pain score indicative of the total physical and mental pain that the user is experiencing;
selecting an interactive content option from a plurality of interactive content options based on said user temporal profile to improve at least one of said one or more temporal user scores, each of the plurality of interactive content options is associated with an effect on at least one of said one or more temporal user scores; and
outputting said interactive content option to the user or a secondary user.

35. The method of claim 34, comprising recording a response of the user to said output interactive content option and updating at least one of said one or more temporal user scores.

36. The method of claim 34, wherein said user-related data comprises user interaction history data; wherein said user interaction history data comprises recorded responses of the user to each output interactive content option; wherein said user interaction history data comprises engagement profile of the user with each output interactive content option; wherein said engagement profile comprises recording of the physical engagement profile of the user with said interactive content option when said interactive content option is being presented on a touch screen.

37. The method of claim 36, wherein said user interaction history data comprises a plurality of data pieces, each associated with a different time stamp, wherein each data piece is assigned with a respective weight factor for affecting at least one temporal user scores.

38. The method of claim 37, wherein the method comprising updating said respective weight factor assigned to each data piece based on the respective time stamp of said data piece; wherein said updating said respective weight factor comprises applying a time stamp-depended function to said respective weight factor.

39. The method of claim 34, wherein said user related data comprises responses of said secondary user to the output interactive content option.

40. The method of claim 34, wherein said user related data comprises any one of: medical data, psychological data, social data and financial data.

41. The method of claim 34, wherein said one or more temporal user scores comprise at least one of the following: mental capacity score, medical burden score, total pain score, friends and family support score, tendency to share medical condition, tendency to share content, tendency to share financial challenges, tendency to consume services, tendency to create content, level of peace, level of control, urgency to create legacy, level of meaning, remaining time perception, level of fear of death, and home treatment trending score, readiness to change location of care.

42. The method of claim 34, wherein said selecting comprises selecting an interactive content option that is associated with an effect on one or more desired temporal user scores.

43. The method of claim 34, wherein said plurality of interactive content options comprise range-based interactive content options that are allowed for selection upon being in a defined range of one or more temporal user scores; wherein the method comprising updating the defined range of each of said range-based interactive content based on said user temporal profile.

44. The method of claim 34, comprising assigning one or more interactive content option weight factors affecting each temporal user scores to at least one interactive content option;
wherein the method further comprising updating said interactive content option weight factors according to said user temporal profile.

45. The method of claim 44, comprising assigning a variance parameter to at least one interactive content option, said variance parameter indicative of the variance of interactions of a population of users with said interactive content option; wherein said assigning one or more interactive content option weight factors comprises applying a variance function dependent on said variance parameter; wherein said variance function is further dependent on said user temporal profile.

46. The method of claim 34, wherein said interactive content options comprise questions or questionnaires and recommendations for actions or for services; wherein said recommendations comprise palliative treatment suggestions; wherein said palliative treatment suggestion is triggered following identification of reaching a threshold of the temporal score of readiness to change location of care.

47. The method of claim 34, wherein said interactive content options comprise tasks for the user.

48. The method of claim 34, comprising receiving general medical-related data indicative of medical statistics, wherein said selecting is further performed based on said general medical-related data.

49. The method of claim 34, comprising receiving location-related data indicative of data related to specific locations, wherein said selecting is further performed based on said location-related data.

50. A system for interacting with a user having a medical condition, comprising:
at least one processing circuitry configured for:
receiving user-related data;
analyzing said user-related data for generating a user temporal profile, said user temporal profile comprises one or more temporal user scores, said temporal user scores comprises at least one of mental capacity score indicative of the capacity of the user for facing with or taking decision on a certain topic, and total pain score indicative of the total physical and mental pain that the user is experiencing;
selecting an interactive content option from a plurality of interactive content options based on said user temporal profile to improve at least one of said one or more temporal user scores, each of the plurality of interactive content options is associated with an effect on at least one of said one or more temporal user scores; and
outputting said interactive content option to the user or a secondary user.

51. The system of claim 50, comprising an output unit for outputting said interactive content option to the user or a secondary user.

52. The system of claim 50, wherein the at least one processing circuitry is configured to perform the method of claim 34.

53. A non-transitory computer-readable medium storing instructions that, when executed by one or more processors, cause one or more processors to perform the method of claim 34.
